(19) United States
(12) Patent Application Publication
(10) Pub. No.: US 2019/0232052 A1
(43) Pub. Date: Aug. 1, 2019

(54) METHOD AND APPARATUS FOR FACILITATING ARTIFICIAL VISION

(71) Applicant: NEWSOUTH INNOVATIONS PTY LIMITED, Sydney, NSW (AU)

(72) Inventors: Alejandro Barriga-Rivera, Randwick (AU); Tianruo Guo, Roseberry (AU); Gregg Jørgen Suaning, Lisarow (AU); John W. Morley, Blaxland (AU); Nigel Hamilton Lovell, Coogee (AU)

(21) Appl. No.: 16/312,366

(22) PCT Filed: Jun. 23, 2017
(86) PCT No.: PCT/AU2017/050645

§ 371 Date: Dec. 21, 2018

(30) Foreign Application Priority Data
Jun. 23, 2016 (AU) 2016902473

(51) Int. Cl. A61N 1/05; A61N 1/36

(52) U.S. Cl. CPC A61N 1/0543; A61N 1/36125; A61N 1/36135; A61N 1/36189

(57) ABSTRACT

The present invention relates to a method and apparatus for facilitating artificial vision using retinal electrical neuro-stimulation. Retinal ganglion cells are stimulated at two different sites in order to elicit better visual percepts.

Primary stimulation is implemented at first site on the retina. Secondary stimulation is applied in the vicinity of the optic disc or optic nerve. The secondary stimulation modulates the signals elicited by the primary stimulation.

### FIELD OF THE INVENTION

The present invention relates to a method and apparatus for facilitating artificial vision and, particularly, but not exclusively, to a method and apparatus for facilitating artificial vision using retinal electrical neuro-stimulation.

### BACKGROUND OF THE INVENTION

The natural functioning of a healthy human eye involves receiving light through the eye-lens, generating neural messages at the retina and sending the neural messages to the brain. Light entering the retina triggers a photochemical reaction in the photoreceptors of the retinal tissue (i.e. cones and rods). Neural responses are transmitted in the retinal neurons, via the optic nerve to the brain. The brain processes these signals to produce meaningful visual percepts (vision).

There are many conditions which can result in the failure of human vision. Attempts have been made to stimulate retinal ganglion cells with signals processed from image sensors, in order to reproduce vision.

There are two main types of retinal ganglion cells (RGCs), On-type and Off-type and they respond differently to light stimulation. The response is complex, but generally speaking, the onset of light stimulation produces a transient burst firing of the On-type cells which will remain sustained during photic stimulus. Off-type cells will remain inactive until the photic stimulus stops. These cells then respond with a sustained burst of action potentials.

It is known that high frequency electrical stimulation (HFS) of the retinal tissue can trigger differential responses in both ON brisk transient and OFF brisk transient RGCs. Although the response of the RGCs following light stimulation is understood, to date their electrical stimulation has not been able to reproduce neural encoding of visual stimuli that result in wholly meaningful visual percepts.

### SUMMARY OF THE INVENTION

In accordance with a first aspect, the present invention provides a method of facilitating artificial vision, comprising the steps of stimulating retinal on-type and off-type cells, and affecting resulting neural responses of the cells to reproduce more natural on-type and off-type cell behaviour.

In an embodiment, the step of stimulating comprises the step of applying a primary stimulating signal to stimulate the retinal On-type and Off-type neurons. The step of affecting comprises providing a secondary stimulating signal to affect the neural responses.

In an embodiment, the primary stimulating signal and secondary stimulating signals are applied at different stimulating sites in the visual system.

In an embodiment, the primary stimulating signal is applied at the retina. In an embodiment, the secondary stimulating signal is applied in the vicinity of the optic nerve where the neural axons gather together. The secondary stimulating signal may be applied proximate or at the optic disc, or at the optic nerve.

In an embodiment, the primary stimulating signal is applied at a proximal portion of a retinal ganglion cell. It may be applied at the soma or initial segment of the axon. In an embodiment, the step of affecting comprises providing a secondary stimulating signal to the retinal ganglion cells distal of the primary stimulating location.

In an embodiment, the secondary stimulating signal modulates the neural responses elicited in the neural axons by the primary stimulating signals.

Advantageously, in an embodiment, the primary stimulus and secondary stimulus enables mimicking of the behaviour of a healthy retina by establishing On and Off responses in isolation. Advantageously, by applying this form of stimulation, the applicants believe that a more physiologically realistic encoding of visual stimuli can be achieved.

In an embodiment, the stimulation applied by the primary and secondary signals is electrical stimulation applied by electrodes positioned at primary and secondary stimulation sites.

In an embodiment, the number of and distribution of electrodes at the primary stimulation site and secondary stimulation site may be varied to influence spatial application of the primary and secondary signals.

In embodiments the primary group of electrodes may be arranged in a rectilinear array, a hexagonal mosaic or octagonal mosaic. They may be distributed randomly or arranged in concentric circles or in any other pattern.

In an embodiment, the secondary group of electrodes may be arranged in an arcuate form near the optic disc or any arrangement or pattern or as cuff about the optic nerve.

In an embodiment, the electrode return configuration may be selected to influence the spatial application of the signals.

In an embodiment, the primary stimulating signal and secondary stimulating signal are delivered sequentially.

In an embodiment, the time periods of the primary and the secondary stimulating signals may be varied to vary stimulation. Further, a time period between application of the primary stimulation signal and secondary stimulation signal may be varied.

In an embodiment, the method comprises the further step of monitoring the signals produced by the stimulation to determine the effect of the stimulation. In an embodiment, the voltage waveforms and the neural responses of the tissue are monitored.

In an embodiment, the method comprises the further step of monitoring local field potentials evoked by the application of the signals, and using this to adjust stimulation.

In accordance with a second aspect, the present invention provides an apparatus for facilitating artificial vision, comprising a stimulator arrangement arranged to stimulate retinal on-type and off-type cells to elicit neural responses, and an affecting arrangement arranged to affect the neural responses to reproduce more natural on-type and off-type cell behaviour.

In accordance with a third aspect, the present invention provides a method of facilitating artificial vision, comprising the steps of stimulating retinal ganglion cells, and affecting resulting neural responses of the cells.

In an embodiment, the step of stimulating comprises the step of applying a primary stimulating signal to a proximal portion of a retinal neural ganglion and applying a secondary stimulating signal to effect the neural responses produced by the primary stimulating signal.

This aspect of the invention may have any or all of the features of the aspects of the invention discussed above. In accordance with a fourth aspect, the present invention provides an apparatus for facilitating artificial vision, comprising a stimulator arrangement arranged to simulate retinal ganglial cells to elicit neural responses, and an affecting arrangement arranged to affect the neural responses.

In an embodiment, the stimulator arrangement is arranged to apply a primary stimulating signal to a proximal portion of a retinal ganglial cell, and a secondary stimulation signal to a distal portion of a retinal ganglial cell, to affect the neural response produced by the primary signal.

In accordance with a fifth aspect, the present invention provides a computer program, comprising instructions for controlling a processor to implement a method in accordance with the first or fifth aspects of the invention.

In accordance with a sixth aspect, the present invention provides a computer readable medium, providing a computer program in accordance with the fifth aspect of the invention.

In accordance with a seventh aspect, the present invention provides a data signal, comprising a computer program in accordance with the first aspect of the invention.

### BRIEF DESCRIPTION OF THE DRAWINGS

Features and advantages of the present invention will become apparent from the following description of embodiments thereof, by way of example only, with reference to the accompanying drawings in which:

FIG. 1 shows a block diagram illustrating a an apparatus for facilitating artificial vision, in accordance with an embodiment of the present invention;

FIGS. 2 to 5 are diagrams illustrating examples of stimulatory implants comprising primary and secondary groups of electrodes implanted in the retina, in accordance with embodiments;

FIG. 6 is a representation illustrating sequential stimulation delivered at primary and secondary groups of electrodes implanted in the retina, in accordance with embodiments;

FIG. 7 shows examples of stimulating waveforms with different types of modulation schemes, in accordance with embodiments;

FIG. 8 shows an example of a model showing natural RGC encoding following engineered electrical stimulations.

### DETAILED DESCRIPTION OF EMBODIMENTS

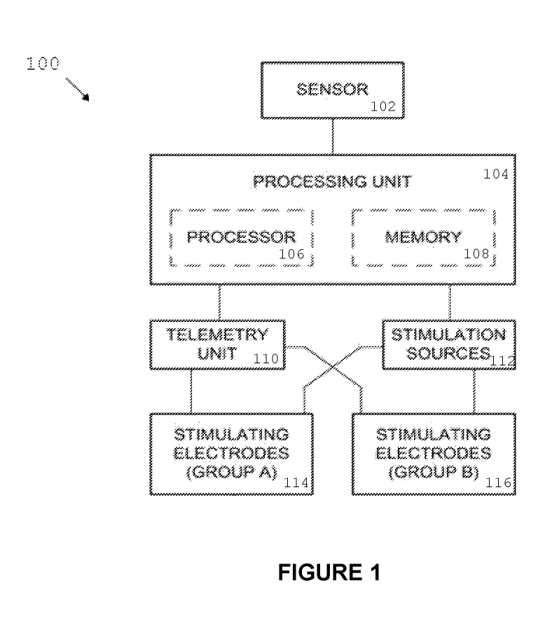

An apparatus in accordance with an embodiment of the present invention is broadly illustrated in FIG. 1. It comprises a processing unit 104 and stimulation sources 112. The processing unit 104 and stimulation sources 112 form a stimulating arrangement for providing primary stimulation signals to stimulate retinal On-type and Off-type neurons, to elicit neural responses in them. The processing unit 104 and stimulation sources 112 also form an affecting arrangement, arranged, in this example, to generate secondary stimulation signals to affect the elicited neural responses. The apparatus in this embodiment also comprises an image sensor 102, for producing image signals which are processed by the processing unit 104 to operate the stimulation sources to provide the stimulation signals.

In more detail, the image sensor 102 in this embodiment (a digital camera) captures visual information in the form of various frames. The image sensor 102 can be a camera or any other device which is capable of capturing visual information in the form of images. The processing unit 104 receives the visual information through image sensor 102. The processor 106 acquires, digitises and processes a series of frames and stores these in the memory unit 108. After processing, a series of stimulating waveforms are delivered through the plurality of stimulating sources 112 (current sources in this embodiment) to two groups of stimulating electrodes 114, 116. The parameters of the stimulating waveforms are correlated to the visual information captured by the image sensor 102. The primary group of electrodes 114 is placed in close proximity to the retinal neural cells and these will be used to deliver a series of primary stimulation waveforms which will recruit target retinal cells. These primary stimulation waveforms will electrically stimulate the RGCs to generate neural responses. The secondary group of electrodes 116 is distributed in the vicinity of the optic disc, where the axons of the RGC converge to form the optic nerve. These electrodes 116 operate as an arrangement which affects the primary neural responses by delivering a series of secondary stimulation waveforms. This modulates the neural responses generated by the primary stimulation of the retina. The modulated neural responses are arranged to more closely replicate the natural neural encoding of light stimuli of On-RGCs and Off-RGCs. The modulated neural responses are carried through the optic nerve to the brain of the patient. In the patient's brain, these responses produce a meaningful visual perception of the real world captured through the image sensor 102.

The apparatus 100 also comprises a telemetry unit 110. The telemetry unit is arranged to measure impedance and local field potentials at the stimulating sites, following electrical stimulation of the On-type and Off-type cells. This information can be used to “tune” the stimulation (see later).

In an embodiment, an implant comprising the primary group 114 and the secondary group 116 of stimulating electrodes is installed in patient's eye. The implant is a device capable of communicating with external electronics for example with the stimulation sources 112 and the telemetry unit 110. The implant can be powered by the processor 106 through a wireless link e.g. radiofrequency induction. The processor 106 sends information to the implant to deliver the primary and secondary simulation signals at target sites in accordance with the visual information received from the image sensor 102.

A number of different electrode arrangements and patterns may be used at the primary stimulating sites and secondary stimulating sites. Different electrode configurations (type of electrode) may also be utilised.

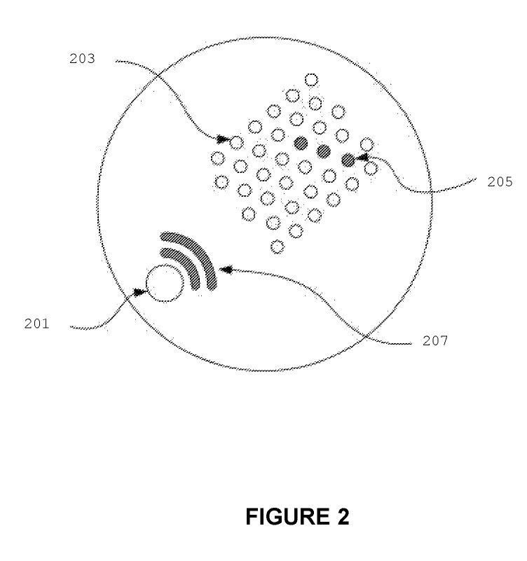

Referring now to FIG. 2, it shows an example of an embodiment of the present invention where the primary group of electrodes is arranged in a substantially hexagonal mosaic near the retinal cells and the secondary group of electrodes is arranged proximate or at the optic disc in the form of two concentric ring sections. The RGC axons tend to run approximately radially, converging to the optic disc to form the optic nerve. Placing the secondary stimulating electrodes at or near the optic disc or at or near the optic nerve is advantageous, as this is where all the axons converge. The neural responses elicited by the primary stimulus may therefore be precisely affected by secondary stimulating electrodes positioned at these sites.

In this diagram, 201 represents the optic disc, 203 and 205 represent inactive and active electrodes in the primary group of stimulating elements respectively. A primary electrode is “active” if it is being stimulated by the primary stimulation signal. This will depend on the signals generated by the image sensor 102 and the waveforms generated by the processing unit 104 to operate the stimulation sources 112. The active electrodes effectively represent the effect of the image being sensed by a sensor 102. The primary stimulating electrodes 203, 205 are arranged in a rectilinear array in this embodiment, following a hexagonal mosaic. The secondary stimulating electrodes 207 are arranged as two concentric ring sections.

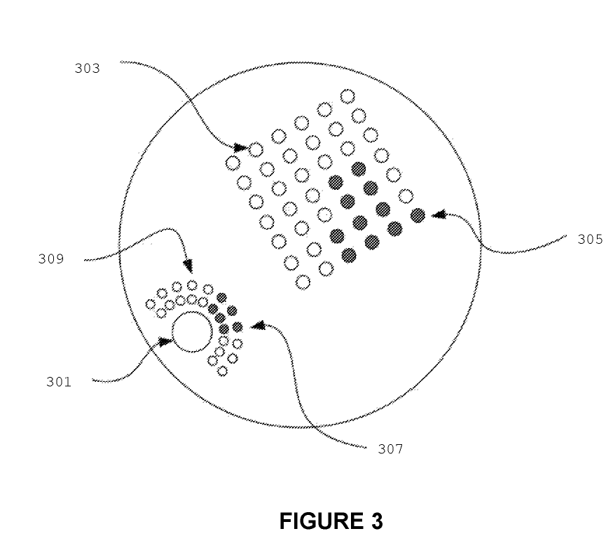

FIG. 3 shows an alternative electrode arrangement. The primary group of electrodes is arranged in a rectilinear pattern near the retinal cells. Electrodes 305 are shown active, and electrodes 303 inactive. It will be appreciated that which electrodes are active and inactive depends on the stimulation applied. A secondary group of electrodes is distributed around the optic disc 301 in the form of two concentric circular section arrangements to allow improved spatial selectivity. This arrangement allows spatial selectivity of the secondary waveforms so as to be able to activate 307 or inactivate 309 a sub-group of them.

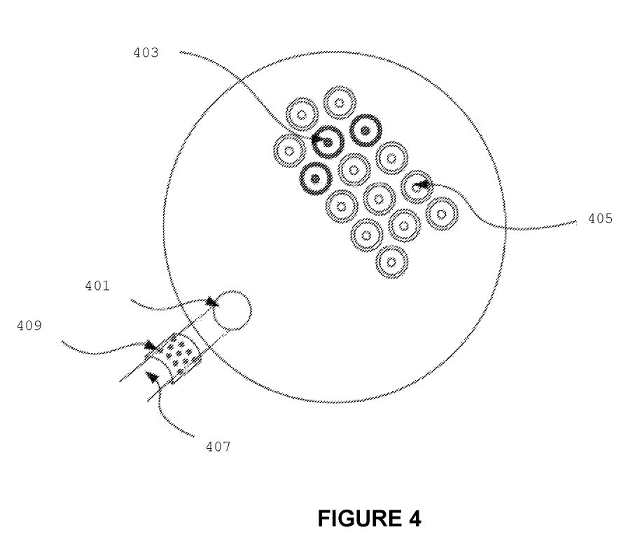

FIG. 4 shows a further example of an embodiment of an electrode configuration. A primary group of electrodes (403 active, 405 inactive) are arranged in a hexagonal mosaic configuration. The secondary group of electrodes 409 are arranged as a cuff about the optic nerve 407.

In this embodiment, the type of electrodes used as the primary stimulating electrodes are of a concentric configuration. This configuration may be used to achieve focus to electrical fields for the primary electrodes.

The configuration for the electrode may be selected, as well as the electrode distribution patterns.

The electrodes may be distributed in any pattern, hexagonally, octagonal pattern, concentric circles or any other pattern. They may be randomly distributed.

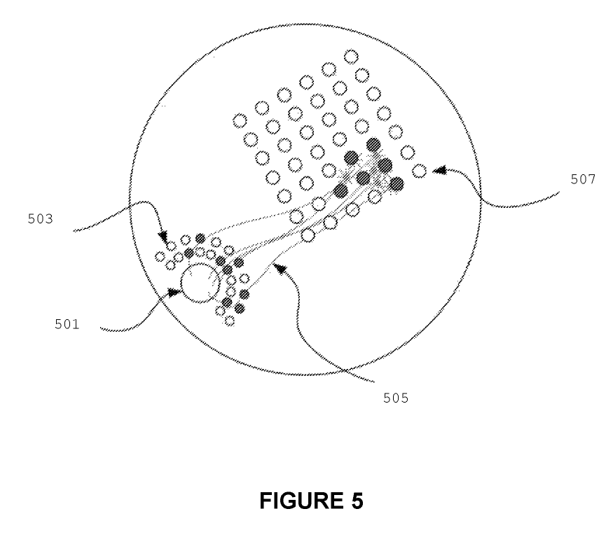

As mentioned previously, both groups (primary and secondary) of electrodes can be connected to a telemetry unit (see FIG. 1) that allows monitoring of the electrode-tissue impedance. Referring to FIG. 5, the telemetry unit in conjunction with the plurality of electrodes and the stimulation sources can be used to record the evoked local field potentials: electrodes within the first group can be activated while the electrodes of the second group can be used to record the evoked potentials and vice versa. This allows for appropriate adjustment to the stimulation strategy for each individual patient.

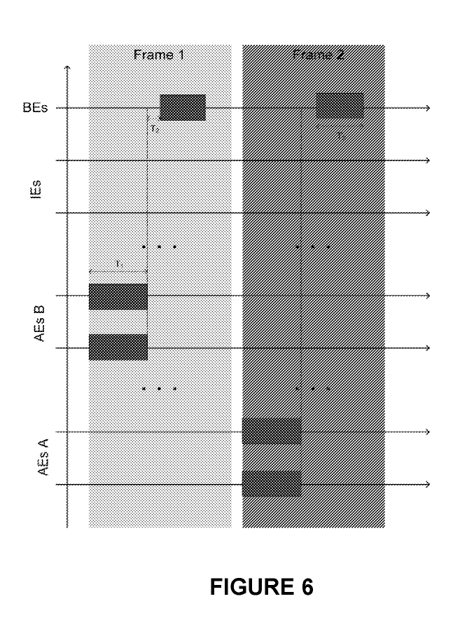

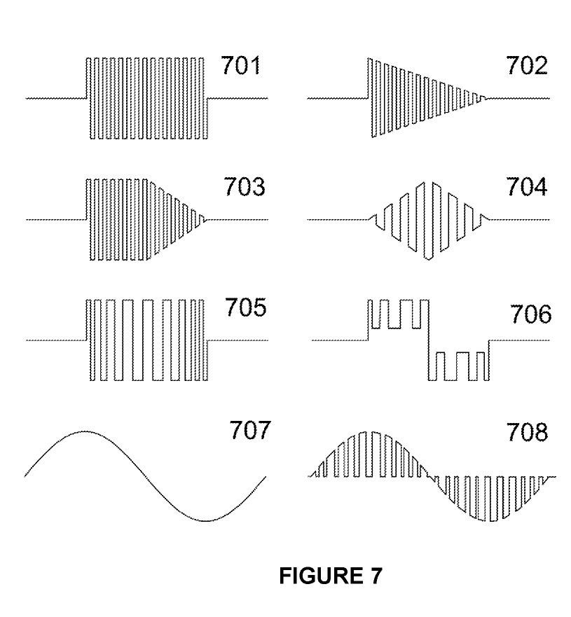

FIG. 6 is a diagram representing a sequential stimulation pattern delivered at the primary and secondary groups of electrodes implanted in the retina to allow mimicking physiological responses of visual stimuli by injecting alternating electric current. To achieve this, a sequence of waveforms is delivered at two different sites in the visual system. A sub-group of electrodes in the primary site is activated in order to elicit the perception correlated with a given visual scene, that is, a frame captured by the image sensor. Afterwards, a sub-group of electrodes in the secondary site is activated following the previous stimulus to modulate the travelling burst response. In FIG. 6, a group of active electrodes in the primary site (AEsB) deliver waveforms that are correlated to the visual scene in Frame 1. Next, a series of waveforms are delivered through active electrodes at secondary site (BEs) to modulate the response. During frame 2, the active group of electrodes represents a different scene and therefore may change (AEsA). Likewise, this is followed by a series of waveforms delivered at the secondary site. In the diagram, T1 represents the duration of the stimulus at the primary site and T3 is the duration of the stimulus at the secondary site. Note T2 represents the delay between both waveforms, which can be positive or negative. T₁ and T₂ and T₃ are parameters that may be selected and controlled by the processing unit 104. Varying these parameters can assist in calibrating the apparatus in order to gain good visual percepts. FIG. 7 illustrates exemplary stimulating waveforms having different types of modulation schemes. Both the primary and secondary waveforms, delivered at the primary and the secondary sites, can be described as a high-frequency carrier modulated by a low-frequency envelope. These waveforms can include amplitude- and frequency-modulation of square pulse trains as shown in the examples, 701 to 708. Example waveforms 701 and 702 represent square and saw shape modulation respectively. A combination of both is illustrated in 703 whereas 704 represents a lower frequency using triangular modulation. An example of frequency-modulation is shown in 705, and 706 shows both amplitude- and frequency-modulations combined. A low frequency signal is shown in 707 which can also be used as primary or secondary waveforms. 708 illustrates an example of achieving similar effects as in 707 through high-count pulse trains using charge balance injection. The inter-stimulus time, as described in FIG. 6 by T2, can be also modulated to modify neural encoding. The frequency and modulation schemes and amplitude of the waveforms or other parameters may be selected and adjusted in order to calibrate the apparatus to produce good visual percepts.

### Example

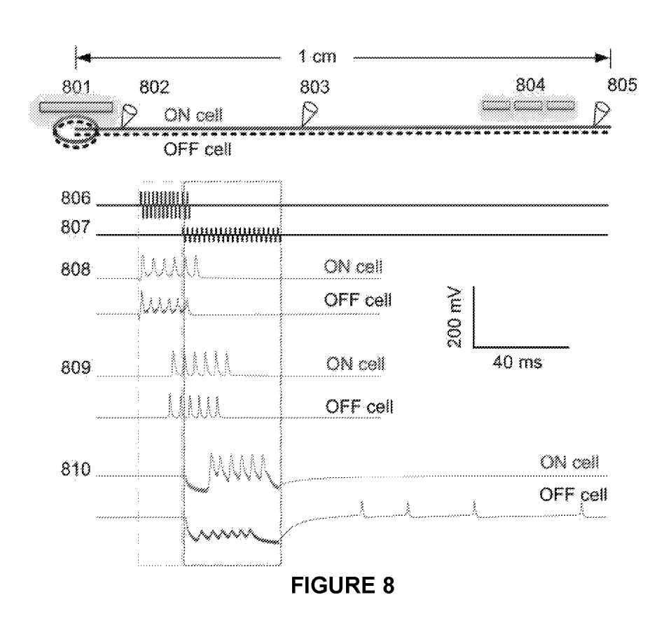

Referring now to FIG. 8, there is shown an example of a computational model of natural RGC encoding following engineered electrical stimulations. A primary electrode (reference numeral 801) of radius 100 μm, used in a monopolar return configuration, was epiretinally positioned 15 μm above the soma. An electrode array (reference numeral 804), arranged hexagonally, was configured following a hexapolar return configuration (radius 15 μm, with 60 μm centre-centre distance) and was positioned distally near the optic disc (reference numeral 805). A primary waveform (reference numeral 806) was delivered near the RGC bodies. A secondary waveform (reference numeral 807) was delivered distally 20 ms after the onset of the primary waveform. Induced responses at the proximal axon (reference numeral 802) following the primary stimulus. Both, On-type and Off-type cells were successfully activated and a series of action potentials were elicited (reference numeral 808). Travelling action potentials (reference numeral 809) were recorded at the middle axon (reference numeral 803). The responses following secondary stimulation at the distal axon 805 indicates that action potentials occurred first in the On-type cells followed by post-offset action potential in the Off-type cells (reference numeral 810). Referring again to FIG. 8, it can be seen that the response induced in the On-cells and Off-cells follow a pattern of a series of spikes. It is believed that the number of spikes is another important parameter that can facilitate correct visual percepts. The primary stimulating waveform and/or second stimulating waveform, in an embodiment, can be varied, to vary the amplitude and number of spikes. This provides another tool by which the apparatus and effect on visual percepts may be calibrated and tuned.

Different electrode return configurations may be used in order to provide spatial selectivity in different embodiments. In a monopolar configuration, the return electrode, generally of larger size than the stimulating electrodes, is placed far from the active electrodes. This will produce a wide spread of the electric current and therefore recruitment of a larger retinal tissue area. The bipolar configuration utilises one of the neighbouring electrodes within the electrode array as a return. This will, to some extent, reduce the spread of the electric field and therefore produce a more contained electrical stimulation, while activating the neural tissue at both sides, the active electrode and the return electrode. In an electrode array where the spatial distribution is in lattice form e.g. hexagonal, then the surrounding electrode configuration acts as a return electrode. That is, with a hexagonal configuration, the hexagonal guard acts a return electrode. Equivalent arrangements may be used for octagonal or square lattices or any other shape lattice. This provides focussed stimulation while increasing activation thresholds. Note that the quasi-monopolar configuration combines a monopolar and a hexapolar approach. This will provide contained stimulation with lower thresholds. Concentric configurations can also be used to replace the hexapolar configurations, to achieve focused electrical fields. These return configurations can be used, in embodiments, in combination with multiplexing techniques to enhance performance.

In the above embodiments, primary stimulating signals are delivered at the retina and the secondary stimulating signals at the optic disc or optic nerve. The invention is not limited to this. The stimulating signals may be delivered anywhere in the visual system.

It will be appreciated that embodiments of the present invention may utilise computer programs to facilitate control of the apparatus. These programs may be in the form of software running an appropriate hardware. They may be in the form of programmable gate arrays (or field programmable gate arrays) or in any other form. Software may be stored in memory, or on other computer readable media, or delivered as signals.

In embodiments, the processing unit that processes images and generates stimulation parameters (for example, the processing unit 104 of FIG. 1) may comprise computer software or firmware for controlling purposes. Further, the implant may include software or firmware that controls the way stimuli are generated and monitors performance of the implant.

In the above described embodiment, the processing unit is external to the human body. In other embodiments, the processing unit may be provided internally. In embodiments, parts of the apparatus may be internal (stimulating electrodes, for example) and parts external. The parts internal and external may be varied, depending on the embodiment. For example, in some embodiments the telemetry unit and stimulation sources circuitry may be internal, in other embodiments they may be external.

It will be appreciated that the apparatus is not limited to the structure disclosed above with reference to FIG. 1. Other apparatus architectures which implement the function of the present invention may be utilised.

In the above embodiment, the retinal ganglion cells are stimulated by electrodes placed proximate the cells. In embodiments, electrodes may stimulate other cells that in turn stimulate retinal ganglion cells. For example, they may stimulate bi-polar cells and/or retina amacrine cells. Primary stimulation may be applied here and then secondary stimulation distal on the RGC.

The above embodiments show and describe various electrode arrays. It will be appreciated that other electrode arrays than shown may be used in other embodiments, and other electrode arrangements.

Embodiments of the present invention have applicability to visual prosthesis. Applicants believe that providing appropriate neural encoding in retinal prosthesis is key to elicit more meaningful visual percepts.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of facilitating artificial vision, comprising the steps of stimulating retinal on-type and off-type cells, and affecting resulting neural responses of the cells to reproduce more natural On-type and Off-type cell responses.

2. A method in accordance with claim 1, wherein the step of stimulating comprises the step of applying a primary stimulation signal to stimulate the retinal On-type and Off-type cells to evoke the neural responses, and the step of affecting comprises providing a secondary stimulation signal to affect the neural responses.

3. A method in accordance with claim 2, wherein the secondary stimulation signal is arranged to modulate the neural response evoked in the neurons by the primary stimulation signals.

4. A method in accordance with claim 2, comprising the further step of varying the frequency and/or amplitude of the primary and/or secondary stimulation signals to vary the stimulus applied.

5. A method in accordance with claim 2, wherein the primary stimulation signal and secondary stimulation signal are applied sequentially.

6. A method in accordance with claim 5, comprising the steps of varying the time periods of the primary and secondary signal, and/or varying a time period between the application of the primary and secondary stimulation signals, in order to affect the elicited perception.

7. A method in accordance with claim 2, wherein the evoked neural response comprise response signals including a plurality of spikes representing local field potentials, and the method comprises a further step of varying the primary and/or secondary stimulation signals to vary the number and/or amplitude and/or frequency of the spikes.

8. (canceled)

9. A method in accordance with claim 8, wherein the primary stimulation signal application site is the retina.

10. A method in accordance with claim 8, wherein the site of application of the secondary stimulation signal is at or proximate the optic disc or at proximate the optic nerve.

11. (canceled)

12. (canceled)

13. An apparatus for facilitating artificial vision, comprising a stimulator arrangement arranged to stimulate retinal on-type and off-type cells to produce neural responses, and an affecting arrangement arranged to affect the neural responses to produce a more natural on-type and off-type cell response.

14. An apparatus in accordance with claim 13, wherein the stimulator arrangement comprises a stimulation source arranged to apply primary stimulation signals to the retinal cells, and the affecting arrangement comprises a stimulation source arranged to apply a secondary stimulation signal to the evoked neural responses from the primary stimulation signals.

15. An apparatus in accordance with claim 13, further comprising a primary group of electrodes arranged to apply the primary stimulation signal, and a secondary group of electrodes arranged to apply the secondary stimulation signal, the primary group of electrodes and secondary group of electrodes being spatially separated in use.

16. An apparatus in accordance with claim 15 wherein the primary group of electrodes are placed at the retina.

17. An apparatus in accordance with claim 16 wherein the primary group of electrodes are arranged in a rectilinear array at the retina.

18. An apparatus in accordance with claim 16 wherein the primary group of electrodes are arranged in a hexagonal mosaic at the retina.

19. An apparatus in accordance with claim 16 wherein the primary group of electrodes are arranged in an octagonal mosaic at the retina.

20. An apparatus in accordance with claim 16, wherein the secondary group of electrodes are arranged at or proximate the optic disc or at or proximate the optic nerve.

21. An apparatus in accordance with claim 20, wherein a secondary group of electrodes are arranged in an arcuate form at or near the optic disc.

22. An apparatus in accordance with claim 20, wherein the secondary group of electrodes are arranged as a cuff about the optic nerve.

23. (canceled)

24. (canceled)

25. An apparatus in accordance with claim 13, further comprising an image sensor for capturing visual scenes.

26. An apparatus in accordance with claim 13, further comprising a processor arranged to process signals from an image sensor to control the stimulator arrangement to provide stimulation signals correlated to the visual information captured by the image sensor.

27. (canceled)

28. An apparatus in accordance with claim 13, further comprising a telemetry arrangement, arranged to record evoked field potentials at the stimulation sites, whereby the recorded field potentials may be used to facilitate adjustment of the stimulation signals.

29. (canceled)

30. An apparatus for facilitating artificial vision, comprising a stimulator arrangement arranged to simulate retinal ganglial cells to elicit neural responses, and an affecting arrangement arranged to affect the neural responses.

31. (canceled)

32. (canceled)

33. (canceled)

34. (canceled)

35. (canceled)
